(19) United States
(12) Patent Application Publication
(10) Pub. No.: US 2024/0216266 A1
(43) Pub. Date: Jul. 4, 2024

(54) TOPICAL PROBIOTIC DELIVERY SYSTEM

(71) Applicant: Jeen International Corp, Fairfield, NJ (US)

(72) Inventors: James Vincent Gruber, Washington, NJ (US); Juan R. Mateu, Ottawa, IL (US); Adam Perle, Lighthouse Point, FL (US); Jed Riemer, Scarsdale, NY (US)

(73) Assignee: Jeen International Corp, Fairfield, NJ (US)

(21) Appl. No.: 18/570,964

(22) PCT Filed: Jun. 14, 2022
(86) PCT No.: PCT/US2022/033500

§ 371 Date: Dec. 15, 2023

Related U.S. Application Data
(60) Provisional application No. 63/210,978, filed on Jun. 15, 2021.

(51) Int. Cl. A61K 8/99; A61K 8/73; A61K 8/81; A61K 8/92; A61K 35/745; A61K 35/747; A61K 36/064; A61K 36/07; A61Q 19/00

(52) U.S. Cl. CPC A61K 8/99; A61K 8/737; A61K 8/8147; A61K 8/922; A61K 35/745; A61K 35/747; A61K 36/064; A61K 36/07; A61Q 19/00; A61K 2800/10

(57) ABSTRACT

A topical probiotic delivery system comprised of (a) at least one quiescent desiccated probiotic, (b) at least one wax, and (c) at least one polymer having a plurality of ionic or ionizable pendant groups. The delivery system is preferably substantially anhydrous and substantially free of preservatives

### FIELD OF INVENTION

Methods and compositions for providing skin benefits, including by helping to maintain skin microbiome balance and/or preventing skin microbiome dysbiosis.

### BACKGROUND OF THE INVENTION

The Human Microbiome Project has characterized five microbial communities: nasal passages, oral cavity, skin, gastrointestinal tract, and urogenital tract. Achieving and maintaining a balance in these microbial communities has been and remains a focus of researchers and product developers in the personal care/cosmetics industry and medicine. One approach is to deliver a variety of beneficial microorganisms to those innately present.

Dermatological research has identified commensal bacterial species on the skin's surface and defined their role in helping prevent alteration of the delicate balance between “good” and “bad” bacteria, which can result in inflammatory skin diseases, including acne, eczema, rosacea and psoriasis, as well as “dry” and “sensitive” skin. See, e.g., health.clevelandclinic.org/what-are-probiotic-skin-care-products-and-do-you-need-them/(bacterial imbalance can cause “flare-ups” and dermatologic problems including: acne, rosacea, eczema, dry and flaky skin). The expanding role of probiotics in the personal care and consumer (non-prescription) skincare industries was described in the review article “Probiotics and Prebiotics in Dermatology”.

J. Amer. Acad. Dermatol., 2014; 71: 4, 814-821. See also, “Probiotics in Skincare: What They Are and How They Work? It's All About Good Bacteria” Elle (UK Edition) (Mar. 27, 2020); “Good-For-You Bacteria: The Best Skin-Care Products Infused with Probiotics” Allure (Oct. 10, 2020); “Why You Should Make Probiotic Skincare Products a Part of Your Beauty Routine” Woman's World (Jul. 29, 2019).

Researchers at L'Oreal have investigated the ability to improve symptoms of atopic dermatitis by topical application of Vitreoscilla filiformis bacterial extract. See, Eur. J. Dermatol. July-August 2006; 16(4):380-4; see also, US Pre-Grant Patent Application Publication No. 20140106016A1 (dandruff) and US Pre-Grant Patent Application Publication No. 20150125412 (hyperseborrhoeic conditions of the scalp).

Collaborative researchers between Gaia AB and the University of Malmo have reported the use of Lactobacillus reuteri DSM 17938 in treating atopic dermatitis Microorganisms 2020; 8: 1026. (doi:10.3390/microorganisms8071026).

Azitra and Bayer developed bioengineered microbiome products using Staphylococcus epidermis, a natural commensal bacteria from the skin microbiome (described as “live biotherapeutic” products) for the treatment of skin diseases and conditions (dry, red, flaky or inflamed skin; sensitive and eczema-prone skin).

Probiotic ingredients are offered by raw materials suppliers including BASF Care Creations (Relipidium© A00265); Symrise (SymReboot™ L19); Chemisches Laboratorium Dr. Kurt Richter GmbH (ProBioBalance NP contains Bifida Ferment Lysate; ProRenew Complex —Lactococcus Ferment Lysate); Bio Component Research (Elmwood Park, NJ; supplier of DermaForce IQ; DermaSpring IQ; Elasitflex IQ; GentleGuard IQ; RoyalTea IQ)

Representative probiotic consumer skincare brands include: AOBiome Therapeutics (which markets Mother Dirt Restorative Skin Probiotics featuring ammonia-oxidizing bacteria); Yun Probiotherapy; and Aurelia Skincare.

The persistence of adenosine triphosphate (ATP), an energy molecule found in all living organisms, including bacteria, fungi, molds, and spores, has been used as a method of measuring cleanliness and hygiene, both in food products, cosmetics and on hard surfaces, including in healthcare environments. See, e.g., L Jimenez, “Molecular diagnosis of microbial contamination in cosmetic and pharmaceutical products: a review” J. AOAC Int. May-June 2001; 84(3):671-5.

Measuring of residual ATP from human skin cells has been used to assess hand hygiene. In 2017, Liceaga et al published an evaluation of the efficacy of ozonated water versus antiseptic hand wash (Hibiclens®) by measuring ATP levels on skin before and after hand washing using Hygiena® SystemSURE™ Luminometer. https://www.beckershospitalreview.com/quality/hand-hygiene-skin-atp-study-hand-washing-with-tap-water-and-soap-vs-ozonated-water-and-soap-vs-antiseptic-and-ozonated-water-vs- antiseptic-and-tap-water.html

U.S. Pat. No. 9,271,924 describes a high-throughput, tiered screening method of identifying ingredients that exhibit prebiotic activity on human skin commensal microorganisms and cosmetic compositions that include such ingredients by an ATP assay (that measures changes in ATP levels).

Prior art methods of assessing skin microbiome imbalance (dysbiosis) include sampling (swab, biopsy, scraping, tape stripping) by 16S rRNA sequencing. Ogai K. et al. “Skin Microbiome Collection by Tape-Stripping” Front Microbiol. 2018 Oct. 2; 9:2362.

Most personal care formulations are comprised of about 70 to 95 percent water, creating significant impacts on the environment. In comparison, anhydrous (or substantially anhydrous) products require substantially less energy not only in terms of manufacturing costs (eliminating energy-intensive heating processes often needed to combine otherwise immiscible ingredients, e.g., water and oil) but also packaging and shipping costs (require less shelf space; weigh less). Eliminating water provides additional benefits. Preservatives, which raise human and environmental health concerns, can be reduced or eliminated in anhydrous products.

“At-home”, “do-it-yourself” personal care products created by adding water are described in U.S. patent application Ser. No. 10/165,369 (filed Jun. 7, 2002) and Ser. No. 11/373,731 (filed Mar. 10, 2006), both now abandoned disclose particles sealed within a self-standing, closed, openable container. When open, a liquid or semi-solid (gel) is added to and mixed with the container contents forming a cosmetic composition. U.S. patent application Ser. No. 10/409,578 likewise describes a ready-to-make cosmetic product comprised of three components: each separately provided: (i) a mixture of cosmetically active ingredients; (ii) an aqueous carrier medium; (iii) a thickening agent. When combined and mixed, the components form a cosmetic cream or lotion.

There has been and remains a long-felt but unmet need for products that deliver viable microorganisms to the skin and ingredients that help support a healthy, balanced skin microbiome, especially in more environmentally responsible anhydrous (or substantially anhydrous) forms. As described and claimed in the present application, the inventive topical probiotic delivery system of the present invention meets this need. See JV Gruber and J Reimer, “Delivering Probiotics by Just-Add-Water Tech”, Personal Care Magazine, 57-61 (June 2022).

### SUMMARY OF INVENTION

A topical probiotic delivery system that helps achieve and maintain skin microbiome balance and/or reduce or prevent skin microbiome dysbiosis comprised of, consisting essentially of, or consisting of (a) a quiescent desiccated probiotic, optionally with one or both of a prebiotic, and/or a lysate of a microorganism, (b) at least one wax and (c) at least one polymer with a plurality of pendant ionizable or ionic moieties selected from carboxyl, hydroxyl, sulfate, thiol, amine, amide, imide, imine, and/or nitrile groups, salts thereof, and combinations of the foregoing, wherein the topical probiotic delivery system is preferably, a flake, a slab, a powder, a paste, a semi-solid, or a slurry.

In a first aspect, the polymer that is an essential component of the topical probiotic delivery system is a polysaccharide. In these embodiments, the at least one wax may be self-emulsifying or non-self-emulsifying. If the at least one wax is not self-emulsifying, the topical probiotic delivery system contains at least one emulsifier.

In another aspect, the polymer that is an essential component of the topical probiotic delivery system has pendant alkali metal carboxylate groups or ammonium carboxylate groups or the polymer is a polyquaternary compound.

The topical probiotic delivery systems of the present invention are preferably “substantially anhydrous” by which is meant it contains less than 5% water, preferably less than 2.5% water, more preferably less than 1% water, still more preferably less than 0.5% water, even more preferably less than 0.1% water.

The topical probiotic delivery systems of the present invention are preferably “substantially preservative-free” by which is meant it contains less than 0.25% of one or more ingredients that retard the growth of and/or kill bacteria, yeasts and/or molds.

The topical probiotic delivery system is “activated” when hydrated, e.g., by contact with a source of water, including a bodily fluid or tissue, optionally with water-soluble ingredients.

### DETAILED DESCRIPTION OF THE INVENTION

A “probiotic” is a live microorganism that is intended to have health benefits when consumed or applied to the body. See, www.nccih.nih.gov/health/probiotics-what-you-need-to-know, accessed on Jun. 15, 2021.

Non-limiting, but preferred examples, of probiotic microorganisms include: (i) Gram positive bacteria selected from the groups of: Lactobacillus; Bifidobacterium; Staphylococcus, preferably Staphylococcus epidermidis; Streptococcus, preferably Streptococcus pyogenes; Cutibacteria, preferably Cutibacteria avidum; (ii) yeasts, such as Saccharomyces boulardii; and (iii) fungi, such as Malasezzia.

A “prebiotic” is a non-digestible food component that selectively stimulate the growth or activity of desirable microorganisms.

The term probiotic as used in the present invention also encompasses “synbiotics”—a product comprised of probiotics and prebiotics.

A “postbiotic” is one or more non-viable bacterial product(s) or metabolic by-product(s) from probiotic bacteria. Postbiotics are typically produced during the fermentation process of creating probiotic bacteria. Dermatology Times, Vol. 41, No. 5 (May 2020)(accessed Jun. 9, 2022) at https://www.dermatologytimes.com/view/role-biotics-skincare.

A “quiescent desiccated probiotic” is a dehydrated, viable microbial organism, preferably selected from bacteria and yeast, that (a) provides skin benefits, including by helping to maintain skin microbiome balance and/or preventing skin microbiome dysbiosis and (b) can be reconstituted to a state of actively growing and dividing when hydrated, preferably with a non-cytotoxic, non-bacteriostatic liquid carrier.

A “viable” microorganism can actively grow and divide. Microbial viability can be determined using imaging assays known to the skilled artisan, including assays that measure membrane permeability and DNA binding.

Bacterial viability is assessed using membrane permeant and impermeant DNA dyes, including Fluorescein Isothiocyanate (FITC) and Sulforhodamine 101 Acid Chloride (also known as “Texas Red©”), and fluorescent labels, including Hexidium Iodide, Promidium Iodide, and Ethidium Homodimer-2.

Yeast cell viability is assessed using FITC and 4′,6-diamidino-2-phenylindole (DAPI) dyes, and Calcofluor White M2R as a fluorescent signal.

“Lysates” of microorganisms are not viable; and, therefore, are not “probiotics” as defined above. Non-limiting examples of lysates include: yeast hydrolysate, biofermented by Lactobacillus strain, commercially available from BASF Care Creations as Relipidium© A00265 (INCI name: Hydrolyzed Yeast Protein; Butylene Glycol; Pentylene Glycol); ProBioBalance NP (Bifida Ferment Lysate) and ProRenew™ Complex (Lactococcus Ferment Lysate) are both available from Chemisches Laboratorium Dr. Kurt Richter GmbH. DERMAFORCE IQ™, a combination of two lysates—Lactobacillus Ferment Lysate and Saccharomyces Lysate, commercially available from Bio Component Research.

“Non-cytotoxic” means microorgansims are viable based on the bacteria and yeast viability tests described below.

“Non-bacteriostatic” means bacteria do not stop reproducing.

“Skin Microbiome Dysbiosis” means a disruption in the balance or distribution of the taxonomic composition of Resident Skin Microbiota and Transient Skin Microbiota.

“Resident Skin Microbiota” are found in the upper parts of the epidermis and congregated in and around the hair follicles and include Staphylococcus, Micrococcus, Corynebacterium, Brevibacterium, Dermabacter, and Malasezzia.

“Transient Skin Microbiota” include Gram-positive Clostridia bacteria and Gram-negative Acinetobacter.

“Skin microbiome dysbiosis” is a change or alteration in the number (also referred to in the art as “microbial bioload”) and/or balance of microorganisms that comprise the skin microbiome. It is expressed as a change in the amount/concentration of bacterial ATP detected by bioluminescence (also known as biofluorescence) expressed in Relative Light Units (RLUs). Change in RLUs can be determined by (i) measuring bioluminescence as expressed in RLUs on a section of human skin (e.g., on the volar forearm); (ii) swabbing the section of skin with an ingredient known to cause skin microbiome dysbiosis (e.g., 3% H₂02), then measuring the reduction in RLUs; (iii) applying a topical probiotic delivery system of the present invention after being reconstituted (hydrated or combined with a non-cytotoxic, non-bacteriostatic liquid carrier) or a finished product (defined below) containing a topical probiotic delivery system of the present invention and measuring the increase in RLUs (relative to baseline and post-H₂02 application).

“Finished Product” means a topically applied cosmetic product, a personal care product (including a soap, body wash, shampoo), or dermatologic products (including one or a combination of active pharmaceutical ingredient(s) list in an Over-The-Counter Monograph issued by the US Food and Drug Administration (FDA) or subject to a New Drug Application (NDA) of Abbreviated New Drug Application (ANDA) approved by FDA, or a foreign (non-US) regulatory agency with equivalent responsibilities for cosmetics, personal care products and dermatologic products).

“Substantially evenly dispersed” means when a product (here, a reconstituted/hydrated topical probiotic delivery system) is applied onto the black portion of an opacity chart (e.g., Form 2A, from Leneta Company, Manwah, NJ) and spread into a film having a thickness of approximately 0.0015 inches using a film applicator (e.g., from BYK Gardner, Columbia, Maryland) streaks, agglomeration, and/or other signs of unevenness are not visibly apparent without the aid of magnification, and the product does not feel grainy or rough to the touch.

Hyaluronic acid (“HA”), also known in the art as hyaluronan, a mucopolysaccharide having repeating units of two disaccharides, D-glucuronic acid and D-N-acetylglucosamine.

HA polymer chains are comprised of repeating disaccharide monomers that attach to each other through beta-1,4 glycosidic bonds, and can have a molecular weight ranging from 1 kDa to over 2,000 kDa.

Within the scope of the present invention disclosure HA includes hyaluronic acid and alkaline or alkaline-earth salts of hyaluronic acid, with sodium hyaluronate (NaHA) being a preferred salt of HA. NaHA may be of natural origin (e.g., extracted from rooster's combs) or fermented (derived from yeast and mold).

“Gums” are polysaccharides exuded by plants that are gelatinous when moist but hardens on drying to form a resinous substance. Gums can be exudates directly from plants or aqueous solutions or suspensions of the exudates, exudates that have undergone fractionation (e.g., filtration or centrifugation), thermal treatment, spray drying, enzymatic treatment or chemical derivatization. Non-limiting examples of gums include Xanthan Gum, Astragalus Gummifer (Tragacanth) Gum, Cyamopsis Tetragonoloba (Guar) Gum, Carob Gum (also known as Carob Seed Gum, Carob Bean Gum, Locust Bean Gum), Gum Arabic (from Acacia senegal or Acacia seyal), Juniperus phoenicea Gum Extract, Tara Gum, Karaya Gum, Ghatti Gum, Cherry Gum, Apricot Gum, Tamarind Gum, Mesquite Gum, Larch Gum, Psyllium, or Fenugreek Gum can also be used.

“Carrageenans” are polysaccharides extracted from red seaweed (Rodophyceae) and includes its salts Calcium Carrageenan, Potassium Carrageenan and Sodium Carrageenan.

“Alginates” are extracts from brown algae (Phaeophyceae) having linear chain structures of 3-D-mannuronic acid and α-L-glucuronic acid. Alginates can be homopolymeric sequences of mannuronic acid (M blocks) homopolymeric sequences of glucuronic acid (G blocks) and mixed sequences of mannuronic acid and glucuronic acid (MG blocks). Common algal sources of alginates include Laminaria digitata, Ecklonia maxima, Macrocystis pyrifera, Lessonia nigrescens, Ascophyllum nodosum, Laminaria japonica, Durvillea antartica, Durvillea potatorum and Laminaria hyperborea. As used in describing the present invention, “alginates” can include salts of alginic acid.

“Pectins” are hetero-polysaccharides composed of two main domains, both of which contain α-D-galacturonic acid (GalA) residues:a homogalacturonan domain made up of α-(1→4) linked α-D-GalA residues, and a rhamnogalacturonan I domain with a backbone made up of repeating units of α-(1→4) linked α-D-GalA and α-(1→2) linked α-L-rhamnose. Commercial pectins are categorized based on degree of methyl esterification: “low”—less than 50% methyl ester groups; and “high”—50% or more methyl ester groups.

In a first group of preferred embodiments, the topical probiotic delivery system of the present invention consists essentially of (i) a polysaccharide which may be anionic, cationic, amphoteric or chemically-modified and (ii) at least one wax. If the polysaccharide-containing topical probiotic delivery system does not contain at least one self-emulsifying wax, it also contains at least one emulsifier.

Anionic polysaccharides include hyaluronan, alginates, carrageenans, gums, and pectin, each defined above.

Cationic polysaccharides include of chitosan, cationic guar gum, cationic hydroxyethylcellulose Amphoteric polysaccharides or chemically-modified polysaccharides include carboxymethyl-chitosan, N-hydroxy-dicarboxyethyl-chitosan, cetyl hydroxyethylcellulose, guar hydroxypropyl trimonium chloride; 2-hydroxy-3-(trimethylammonio) propyl ether chloride; locust bean hydroxypropyl trimonium chloride; and Casalpina spinosa hydroxypropyl trimonium chloride.

Especially preferred, but non-limiting, polysaccharides suitable for use in topical probiotic delivery systems are sodium alginate, carrageenan, carboxymethyl cellulose, xanthan, starches, cationic guar, and cationic inulin.

Non-limiting examples of a polysaccharide and wax(es) that are essential components of the topical probiotic delivery system in accordance with the first aspect of the invention are:
(i) Stearyl Alcohol, Cetearyl Alcohol, Hydroxypropyl Guar, Cetyl Alcohol, Polysorbate 60 and Glyceryl Stearate; and
(ii) Cetearyl Alcohol, Glyceryl Stearate, Dehydroxanthan Gum, and Cetearyl Glucoside.

In certain embodiments, a polysaccharide-based probiotic delivery system may be used to deliver the quiescent, desiccated probiotic to mammalian mucosal tissue (e.g., oral, nasal, or urogenital).

In a second group of embodiments, the polymer that is an essential component of the topical probiotic delivery system has pendant alkali metal carboxylate groups or ammonium carboxylate groups. One particularly preferred polymer within the scope of this embodiment is sodium polyacrylate.

Non-limiting examples of the combination of polymers with pendant alkali metal carboxylate groups or ammonium carboxylate groups and wax(es) that are essential components of the topical probiotic delivery system in accordance with the second aspect of the invention include:
(i) Sodium Polyacrylate and one of the following waxes: Beeswax; Carnauba Wax; Sunflower Seed Wax; or Synthetic Wax;
(ii) Cetearyl Alcohol, Sodium Polyacrylate and Polysorbate 60;
(iii) Cetearyl Alcohol and Stearic Acid in combination with: (i) Ceteareth-20 and Sodium Polyacrylate or (ii) Glyceryl Stearate, PEG-100 Stearate and Sodium Polyacrylate; (iv) Cetyl Alcohol, Glyceryl Stearate, Stearic Acid, Glycol Stearate, and (i) Sodium Acrylate/Sodium Acrylol Dimethyl Taurate Copolymer or (ii) Stearamide AMP, Carbomer;
(v) Cetyl Alcohol, Glyceryl Stearate and Caprylic/Capric Triglyceride in combination with (i) Sodium Acrylate/Sodium Acrylol Dimethyl Taurate Copolymer or (ii) Sodium Polyacrylate and Polysorbate 80;
(vi) Sodium Polyacrylate/Sodium Acrylol Dimethyl Taurate Copolymer and (i) Sunflower Seed Wax; (ii) Cetyl Alcohol, Glycol Stearate, Glyceryl Stearate, Caprylic/Capric Triglyceride; or (iii) Polysorbate 80, Glyceryl Stearate, Caprylic/Capric Triglyceride.
(vii) Polyethylene and Sodium Polyacrylate, together, and in further combination with (i) Polyvinylpyrrolidone, (ii) Polysilicone-11; or (iii) Polysorbate 80 and Polysilicone-11; and
(viii) Synthetic Wax and Sodium Polyacrylate; alone and, in further combination with Polysilicone-11.

“Waxes” are lipophilic fatty substances, which are solid or semi-solid at room temperature, that undergo a reversible solid-liquid change of state, preferably having a melting point of from about 30° C. to about 165° C., more preferably from about 35° C. to 100° C. But waxes having a melting point of greater 100° C., as well as waxes having a melting point at or below room temperature (25° C.) can be used in certain embodiments of the present invention. In accordance with this definition, waxes include fatty acids, fatty alcohols, esters of fatty acids (including esters of fatty alcohols and fatty acids), and hydrocarbons.

In a third group of embodiments, the topical probiotic delivery system of the present invention consists essentially of (i) a quiescent desiccated probiotic, optionally with one or both of a prebiotic, and/or a lysate of a microorganism, (ii) a polyquaternary compound (preferably, Polyquaternium 7 or Polyquaternium 37), and (iii) a wax.

One non-limiting example of the combination of a polyquaternary compound and wax that are essential components of the topical probiotic delivery system in accordance with third aspect of the present invention is Cetearyl Alcohol, PEG-150 Distearate and Polyquaternium-37.

Topical probiotic delivery systems of the present invention can include a natural wax, a synthetic wax, a combination of natural waxes, a combination of synthetic waxes, or a combination of natural wax(es) and synthetic wax(es).

The wax(es) can be non-micronized, micronized, or self-emulsifying.

Preferred but non-limiting waxes include sunflower wax, candelilla wax, olive wax, and carnauba wax.

Preferred but non-limiting examples of waxes that are or can be made to be self-emulsifying include polyethylene, paraffin, glyceryl monostearate, and polyglyceryl-3 stearate.

In certain embodiments, the wax is a micronized wax, and preferably has a mean particle size from 5 to 50 microns.

In some embodiments the topical probiotic delivery system is “substantially anhydrous” by which is meant it contains less than 5% free water, preferably less than 2.5% free water, more preferably less than 1% free water, still more preferably less than 0.5% free water, even more preferably less than 0.1% free water. For clarity, “free water” is distinguished from “bound water”—namely, water that is an integral part of the topical probiotic delivery system of the present invention, which cannot be removed without changing its structure or composition.

The topical probiotic delivery system of the present invention is formed by combining at least one quiescent dessicated probiotic, optionally with one or both of a prebiotic and/or lysate with at least one polymer having ionic or ionizable pendant groups and at least one wax using blending or mixing device known to the person having ordinary skill in the art (e.g., V-blender, Ross mill rotating blender, tumble blender, vibrating blender).

Surprisingly and unexpectedly, the quiescent dessicated probiotic remains viable at temperatures of up to 45° C.

Viability of the quiescent, desiccated probiotic after the topical probiotic delivery system of the present invention has been reconstituted is demonstrated by the following test method:
1. Topical probiotic delivery systems of the present invention are stored at 4° C., 25° C., 40° C., and 50° C. for up to four weeks.
2. At one-week intervals (i.e., on the 7^{th}, 14^{th}, 21^{st} and 28^{th} days of storages, the topical delivery systems are removed from storage. 5 grams of the topical probiotic delivery system are combined with 95 grams of water and mixed (750 RPM for 20 minutes) forming a probiotic skin cream.
3. 1 gram of the cream from step 2 is added 9 mL of 3M© Buffered Peptone Water Broth ISO (“3M Broth”) and vortexed until fully dissolved. Three further serial dilutions, each in a 1:9 ratio, are prepared.
4. The fourth serial dilution is then plated on a 3M© Petri Film Lactic Acid Bacteria Count Plate (“3M Films”) which is then incubated for 24 hours at about 32° C.
5. Microorganism bioload is quantified at the end of the 24-hour incubation period. Viability is confirmed when at least 50 CFU/gram, preferably at least 100 CFU/gram, 1,000 CFU/gram after incubation are counted.

At the time of use, the topical probiotic delivery system is reconstituted/hydrated by combining with a liquid carrier, preferably water or a mixture of water with one or more water soluble ingredients. Non-limiting examples of water-soluble ingredients that can be included in the liquid carrier include glycerin, polyglycerin fatty acid esters having a polyglycerin of 2-20 units and at least one branched fatty acid residue of 8 to 22 carbons or lower glycols (i.e., having less than 6 carbons), but excluding ethanol and isopropanol.

In preferred embodiments—those that are substantially preservative-free—the topical probiotic delivery system is reconstituted/hydrated into a finished product (e.g., emulsion) in an amount sufficient for a single application.

In other embodiments, the topical probiotic delivery system is reconstituted/hydrated into a finished product in an amount sufficient to be stored and used for multiple applications.

One aspect of the present invention is directed to “at-home” or “do-it-yourself” probiotic skincare compositions (e.g., creams, lotions, serums), in which a topical probiotic delivery system is stored in a separate container (e.g., packet or sachet) that is ready-to-mix with water or other non-cytotoxic, non-bacteriostatic liquid carrier.

In one set of embodiments, the topical probiotic delivery system is stored in a single sachet (or packet) divided into two sections, with a seam or other separator between the two sections—a first section comprised of (i) quiescent dessicated probiotic(s) and/or synbiotic(s) and/or lysate(s), (ii) at least one polymer with a plurality of pendant ionizable or ionic moieties and (iii) at least one wax; and a second section comprised of one or more oils, emulsifiers, and other “active ingredients”, as described below.

The topical probiotic delivery system can also be provided in ajar or tube.

The “at home” or “do-it-yourself” probiotic skincare composition (e.g., in a sachet, packet, jar or tube) can be provided as part of kit further comprised of one or more of (i) a measuring implement, (ii) a mixing implement, and/or (iii) a container.

The topical probiotic delivery system can, and in certain embodiments, is heated and mixed to a temperature preferably below 45° C. One such heating/mixing device is described in Pre-Grant US Patent Applications 2021/0106508 and 2021/0106959.

The topical probiotic delivery system of the present invention can contain one or more ingredients that provide one or more cosmetic and/or therapeutic effects when applied to the skin, nails or hair, commonly referred to in the art as “active ingredients”.

“Active ingredients” may be incorporated (i.e., included) in topical probiotic delivery systems of the present invention at a concentration ranging from about 0.001% to about 20% preferably from about 0.005% to about 10% by weight of the powder, more preferably from about 0.01% to about 5% by weight of the powder, and even more preferably from about 0.1% to about 2.0%, and include: agents for the treatment of an inflammatory dermatosis, including acne, psoriasis or rosacea; anti-microbial and anti-fungal actives; anti-itch agents; topical anaesthetics; sunscreens; emollients and skin soothing agents; non-steroidal anti-inflammatory agents; humectants and moisturizing agents, including hyaluronic acid, and glycols; skin barrier protectants; lipids and ceramides, including vegetal-derived oils and butters; exfoliants and desquamatory agents; antioxidants and “counter-aging” agents that reduce the appearance of fine lines and wrinkles, including vitamins, proteins and peptides; skin bleaching and lightening agents; artificial tanning agents.

Preferred but non-limiting examples of vegetal-derived oils and butters include: apricot oil; avocado oil; canola oil; castor oil; coconut oil; cottonseed oil; eucalyptus oil; evening primrose oil; flaxseed oil; grape seed oil; jojoba oil; lavender oil; macadamia nut oil; olive oil; peppermint oil; rice bran oil; safflower oil; sesame oil; soybean oil; sunflower oil; sweet almond oil; tea tree oil; wheat germ oil; cocoa butter; and shea butter.

Preferred vitamins and vitamin derivatives include, but not limited to: ascorbic acid and its esters ascorbyl palmitate and magnesium ascorbyl phosphate; tocopherol and its esters tocopheryl acetate; retinol and its ester, retinyl palmitate; retinaldehyde; panthenol and niacinamide.

Preferred skin brightening ingredients include, but are not limited to: arbutin, hydroquinone, konjac acid, ascorbic acid, magnesium ascorbyl phosphate and ascorbyl glucosamine.

Preferred “counter-aging” agents include, but are not limited to “energizing” ingredients (e.g., caffeine, theophylline, adenosine) and skin exfoliants (e.g., glucosamine, hydroxy acids), and protease enzymes (e.g., bromelain, papain).

Non-limiting examples of plants extracts that may be included in the topical probiotic delivery system of the present invention may be extracted from: acerola; agrimony; aleppo pepper; alfalfa; algae; algaeoy; alkanet root; almond; almond; aloe vera; american ginseng; amla fruit; andrographis paniculata; angelica; anise; apple; apricot; apricot kernel; apricot leaves; arbutus; arnica; arrowroot; artichoke; asafoetida; ascophyllum; ashwanganda; asparagus; astragalus; autolyzed yeast; avocado; balm mint; balm of gilead; balsam canada; bamboo; banana; banana leaves; barberry; barley malt; basil; bayberry bark; bayberry; bay laurel; bearberry; bee balm; beechnut; bee pollen; beet; benzoin; berberis; berberry; bergamot; bilberry; bilberry leaves; birch bark; birch leaves; bistort; bitter melon; bitter orange; bitter orange peel; bittersweet; blackberry fruit; blackberry leaves; black cohosh; black currant; black-eyed susan seed; black henna; black pepper; black raspberry; black rice; black snakeroot; black walnut hull; black walnut leaves; bladderwrack; bloodroot; blue agaveueous ph; blue agave; blueberry; blue corn; blue flag; blue malva; blue vervain; borage; boswellia serrata; brazil nut; broccoli; brussel sprouts; buchu; buckthorn bark; bupleurum falcatum root; burdock root; burnet root; butcher's broom; cabbage; cactus; cactusueous; cactus fresh; calendula; camphor; cantaloupe; capsicum; caraway; cardamom seed; carrot; carrot seed; cascara sagrada; cassia; catnip; cats claw; cedar wood; celandine; celery; chamomile; chaste tree berry; cherimoya; cherry; chestnut; chickweed; chrysanthemum; cinchona; cinnamon; clary sage; cleavers; clove; cocoa; coconut; coffee; coltsfoot; comfrey; comfrey root; condurango; coneflower; coriander; cornflower; corn; cornsilk; cotton seed; cowslip; cranberry; cranesbill; cucumber; cumin seed; daffodil; daisy; damiana; dandelion; date; devil's claw; dill; dong quai; drummond phlox seed; dulse; eggplant; elderberry; elderflower; elecampane; eucalyptus; european centaury; evening primrose; eyebright; fennel seed; fenugreek; feverfew; fig; flaxseed; fo-ti root; frankincense; freesia; fumitory; galanga root; ganoderma; gardenia; garlic; gentian; geranium; ginger root; Ginkgo biloba leaf, Ginkgo biloba; ginkgo nut; ginseng; goji berries; goldenrod; goldenseal; golden seal; gotu kola; grape; grapefruit; grapefruit peel; grapefruit seed; grape leaf, grape seed; grape seedoy; grape skin; green bean; guarana; guava; hawaiian ti; hawaiian white ginger; hawthorn berry; hazelnut; heather; hibiscus; honey; honeydew; hops; horehound; horny goat weed; horse chestnut; horseradish; horsetail; huckleberry; hyacinth; hypericum; hyssop; iceland moss; impatiens; indian cress; indian hemp root; irish moss; isatis root; italian olive; ivy; japanese green tea; japanese knotweed; jasmine; jewel weed; jojoba meal; jujube fruit; juniper berry; kaffir lime leaves; kangaroo paw; kava kava; kiwi; kiwi seed; kola nut; lady's mantle; lady's slipper; laminaria; lavender; lecithin; lemon bioflavonoids; lemon fruit; lemongrass; lemon myrtle; lemon peel; lemon verbena; lettuce; lichen; licorice root; lily of the valley; lime flower; lime fruit; lime peel; linden flower; linden tree; locust bean; lovage root; lupine seed; magnolia bark; mandarin orange; mango; marjoram; marsh mallow; meadowsweet; meyer lemon; milk vetch; millet; mimosa bark; mistletoe; molasses; moringa oleifera seed; mugwort; mulberry bark; mulberry leaf, mullein; mushroom; myrrh; myrtle; neem; neroli; nettle; neutral henna; noni; nutmeg; oak bark; oat; oat flour; oatmeal; olive leaf, onion; oolong tea; orange bioflavonoids; orange blossom; orange fruit; orange peel; orchid; oregon grape; orris root; pansy; papaya fruit; papaya leaves; paraguay tea; parsley; parsley seed; passionflower; passion fruit; patchouli; pau d'arco tea; peach; peach kernel; peach leaves; pea; pear fruit; pellitory; pennyroyal; peony flower; peony root; peppermint; periwinkle; persimmon fruit; persimmon leaf, phellodendron amurense; pineapple; pine bark; pine cone; pink peppercorn; plankton; plantain; plum; plumeria; pomegranate; poppy; poppy seed; potato; prickly ash; prickly pear cactus; prickly pear; psoralea; pueraria; pumpkin; pyrethrum; queen of the meadow; quince seed; quinoa; raspberry; raspberry leaves; red clover blossom; red henna; red sage root; red sandalwood; red vine; rehmannia; rest harrow; resurrection plant; rhatany root; rhubarb root; rice bran; roman chamomile; rooibos; rose; rosehips; rosemary; rosewood; roucouyer seed; royal jelly; sacred lotus; sacred lotus seed; safflower; saffron; sage; sarsaparilla; sassafras; saw palmetto berry; schisandra; sea buckthorn; sea fennel; sea grapes; sea kelp; senega root; sesame seed; shave grass; sheep's sorrel; shiitake mushroom; siberian ginseng; skullcap; slippery elm bark; snakeroot; soap bark; soapwort; solomon's seal; southernwood; soybean; spanish moss; spearmint; spinach; spirulina; star fruit; star gazer; st. john's wort; stone root; strawberry; strawberry leaves; sugar cane; sugar maple; sumach; suma root; sunflower petal; sunflower seed; surinam cherry; sweet flag; szechuan lovage root; tamarind; tangerine; tangerine peel; tansy; taro root; tea; texas bluebonnet seed; thai tea; thistle; thyme; tiger lily; tomato; tormentil; tuberose; turmeric; uva ursi; valerian; vanilla; viburnum; violet; walnut seed; wasabi; watercress; water lily; watermelon; watermint; wheat; wheat bran; wheat germ; wheat grass; white lily root; white nettle; white pond lily; white tea; wild cherry bark; wild cherry fruit; wild mint; willow bark; wintergreen; witch hazel; yam; yarrow; yellow dock; yerba santa; and yucca.

Non-limiting examples of surfactants that can be added to the topical probiotic delivery system of the present invention include: Cocoamidopropyl Betaine; Sodium Cocoyl Isethionate; Sodium Lauroamphoacetate; Sodium Methyl Cocoyl Taurate; Sodium Lauryl Sulfate (SLS); Sodium Laureth Sulfate (SLES). In certain preferred embodiments, the surfactant is neither SLS nor SLES. Surfactants can be cationic (e.g., Stearamidopropyl Dimethylamine).

Emulsifiers that can be included in the topical probiotic delivery system include: oil-in-water emulsifiers (e.g., Ceteareth-20); silicone-in-water emulsifiers (e.g., PEG-12 Dimethicone); water-in-oil emulsifiers (e.g., Polyglyceryl-4 Oleate); water-in-silicone emulsifiers (e.g., PEG/PPG-30/10 Dimethicone or Lauryl PEG/PPG-18/18 Methicone).

Emulsifiers that can be included in the topical probiotic delivery system may be cationic, anionic, non-ionic, or amphoteric.

Two non-limiting but preferred examples of multi-functional, cationic, conditioning emulsifiers that can be included in topical probiotic delivery system of the present invention are Behentrimonium Methosulfate, Behentriumonium Chloride, Stearamidopropyl Dimethylamine, or Cetrimonium Chloride.

The following examples are illustrative. Modifications will be apparent to, and can be readily made by, those skilled in the art without departing from the spirit and scope of the invention.

The scope of the appended claims is not to be limited to the examples.

### Examples 1-5: Topical Probiotic Delivery Systems with Probiotic

| | | Saccharomyces | Lactobacillus | Lactobacillus | |
|---|---|---|---|---|---|
| Example | Polymer/Wax | cerevisiae | plantarum | acidophilus | Malasezzia |
| 1 | Helianthus Annuus | 99.99:0.01 | 99.75:0.25 | 99.5:0.5 | 99.25:0.75 |
| | Seed Wax; Sodium | | | | |
| | Polyacrylate | | | | |
| 2 | Cetearyl Alcohol; | 99:1 | 98.75:1.25 | 98.5:1.5 | 98.25:1.75 |
| | Behentrimonium | | | | |
| | Chloride; | | | | |
| | Polyquaternium-37 | | | | |
| 3 | Cetearyl Alcohol; | 98:2 | 97:3 | 96:4 | 95:5 |
| | Guar Hydroxypropyl- | | | | |
| | trimonium Chloride; | | | | |
| | Citric Acid; Cetearyl | | | | |
| | Glucoside | | | | |
| 4 | Cetearyl Alcohol; | 97.5:2.5 | 96.5:3.5 | 94.5:5.5 | 90:10 |
| | Hydroxypropyl Guar | | | | |
| 5 | Cetearyl Alcohol; | 91:9 | 92:8 | 93:7 | 96:4 |
| | Glyceryl | | | | |
| | Monostearate; | | | | |
| | Hydroxypropyl Guar | | | | |

### Examples 6-8: Topical Probiotic Delivery Systems with Microbial Lysate

| | | | | Lactobacillus |
|---|---|---|---|---|
| | | | | Ferment |
| | | Bifida | Lactococcus | Lysate and |
| | Polymer/Wax/ | Ferment | Ferment | Saccharomyces |
| Example | Probiotic blend | Lysate | Lysate | Lysate |
| 6 | Polymer/Wax/Probiotic | 99.5:0.5 | 99:1 | 98:2 |
| | blend from Example 1. | | | |
| 7 | Polymer/Wax/Probiotic | 97:3 | 96:5 | 95:5 |
| | blend from Example 2 | | | |
| 8 | Polymer/Wax/Probiotic | 94:6 | 93:7 | 92:8 |
| | blend from Example 5 | | | |

### Examples 9-12: Finished Topical Products

Finished topical products can be made by adding a topical probiotic delivery system of the invention, as illustrated by Examples 1-8, to water and combining with other ingredients. Advantageously, such formulations can be made without heating the different phases to achieve a homogenous product.

### Example 9: Probiotic Skin Calming Lotion

| Phase | INCI Name | % |
|---|---|---|
| A | Deionized Water | QS |
| A | Glycerin | 0.9 |
| A | Allantoin | 0.2 |
| A | Citrus Aurantium Bergamia (Orange) Fruit Extract; Maltodextrin | 2.0 |
| A | Topical probiotic delivery system-Example 1 or 2 | 5.0 |
| A | Polysorbate-20 | 0.7 |
| A | Glycereth-26 | 12.0 |
| B | Diisopropyl Sebacate | 1.0 |
| B | DL-alpha-Tocopherol Acetate | 0.2 |
| B | Dimethicone | 1.5 |
| B | Mentha Piperita (Peppermint) Oil | 0.2 |
| B | Caprylic/Capric Triglyceride | 0.5 |

Add Phase A ingredients one at a time with continuous propeller mixing @ 500-700 RPM. Each ingredient is dissolved/dispersed prior to addition of the remaining Phases. Add Phase B ingredients to Phase A one at a time with continuous propeller mixing.

### Example 10—Probiotic Skin Cream

| Phase | INCI Name | % |
|---|---|---|
| A | Glycerin | 3.3 |
| B | Deionized Water | QS |
| B | Water; Sodium Hyaluronate (1.0%) | 1.0 |
| B | Topical probiotic delivery system-Example 3 or 4 | 0.8 |
| B | Polyacrylamide; C₁₃₋₁₄ Isoparaffin; Laureth-7 | 2.5 |
| C | Dimethicone; Bis-vinyl-Dimethicone/Dimethicone Copolymer | 2.4 |

Premix Phase A ingredients in a side vessel with propeller stirring. Add Phase B ingredients to a main vessel and mix with propeller until homogeneous. Add Phase A to Phase B and mix with propeller mixer until homogeneous. Add Phase C to Phase A/B and mix until homogeneous.

### Example 11—Probiotic Skin Moisturizing Serum

| Phase | INCI Name | % |
|---|---|---|
| A | Deionized Water | QS |
| A | Water; Polyvinylpyrrolidone (30%) | 8.0 |
| A | Glycereth-26 | 3.0 |
| A | Water, Sodium Hyaluronate; Citrus Auranifolia; Ruscogenin; | 3.0 |
| | Zingiber Officinale; Hibiscus Sadariffa | |
| A | Sodium Hyaluronate; Hypericum Perforatum; Schizophyllum Grifola; | 3.0 |
| | Chamomile Recutita; Althaea Officinalis | |
| B | Topical probiotic delivery system-Example 5 or 6 | 4.0 |
| C | Bismuth Oxychloride (and Mica; Carmine; Magnesium Stearate | 0.04 |
| C | Nylon-12 | 0.36 |
| C | Mica | 0.05 |
| D | Isopropyl Myristate; Isododecane; | 3.0 |
| | Bis-Vinyl Dimethicone/Dimethicone Copolymer | |
| D | Isononyl Isononanoate | 2.0 |
| D | Squalane | 1.0 |
| D | DL-alpha-Tocopherol Acetate | 0.2 |

In a main mixing vessel add Phase A ingredients and mix well with a propeller mixer. Add Phase B ingredients to Phase A one at a time and mix well until homogeneous. Add Phase C ingredients to Phase A/B mixture and mix well until homogeneous. Add Phase D ingredients to Phase A/B/C mixture and mix well until homogeneous.

### Example 12—Mild Probiotic Skin Cleanser

| Phase | INCI Name | % |
|---|---|---|
| A | Deionized Water | QS |
| A | DL-Panthenol | 0.1 |
| A | Water, Sodium Hyaluronate; Salvia Officinalis; Aesculus | 3.0 |
| | Hippocastanum; Glechoma Hederacea Extract; Saccharomyces Copper | |
| | Ferment; Saccharomyces Selenium Ferment; Saccharomyces Platinum | |
| | Ferment; Saccharomyces Zinc Ferment | |
| A | Glycereth-26 | 1.0 |
| B | Topical probiotic delivery system-Example 7 or 8 | 6.0 |
| C | Disodium Laureth Sulfosuccinate; Sodium Cocoyl Isethionate; | 4.5 |
| | Cocamidopropyl Betaine; Sodium Methyl Cocoyl Taurate | |

Add Phase A ingredients one at a time to main mixing vessel and stir well with a propeller mixer. Add Phase B ingredient to Phase A and mix until homogeneous. Add Phase C ingredient to Phase A/B mixture and mix until homogeneous.

The above examples are meant to demonstrate how topical probiotic delivery systems can be used in various topical preparations to provide skin benefits, including improving barrier function, reducing trans-epidermal water loss, and improving conditions resulting in or caused by skin microbiome dysbiosis. They are not meant to be limiting and other examples of formulations containing the ingredients are possible.

In describing compositions and methods of the present invention, the following conventions are to be understood: Numbers are modified by the term “about.” Numerical ranges are meant to include numbers within the recited range, and combinations of subranges between, the given ranges. For example, a range from 1-5 includes 1, 2, 3, 4 and 5, as well as subranges such as 2-5, 3-5, 2-3, 2-4, 1-4, etc. “At least one” is to be understood to mean one or more, and also includes individual components as well as mixtures/combinations. Unless otherwise specified, all percentages, ratios and proportions are by weight; and all temperatures are in degrees Celsius.

Scientific publications cited above are incorporated, in relevant part, herein by reference. The citation of a document is not, however, to be construed as an admission that it is prior art. To the extent that there is a conflict between the use of a term in this invention disclosure and a document incorporated by reference, the meaning or definition herein will apply.

1. A topical probiotic delivery system comprised of: (a) at least one quiescent desiccated probiotic; (b) at least one wax; and (c) at least one polymer having a plurality of ionic or ionizable pendant groups.

2. The topical probiotic delivery system of claim 1 wherein the at least one polymer having a plurality of ionic or ionizable pendant groups is a polysaccharide and the at least one wax is self-emulsifying.

3. The topical probiotic delivery system of claim 1 wherein the polymer having a plurality of ionic or ionizable pendant groups is a polysaccharide, the at least one wax is not self-emulsifying, and the topical probiotic delivery system further comprises at least one emulsifier.

4. The topical probiotic delivery system of claim 1 wherein the polymer has pendant alkali metal carboxylate groups or ammonium carboxylate groups.

5. The topical probiotic delivery system of claim 4 wherein the polymer having a plurality of ionic or ionizable pendant groups is sodium polyacrylate.

6. The topical probiotic delivery system of claim 1 wherein the polymer having a plurality of ionic or ionizable pendant group is a polyquaternary compound.

7. The topical probiotic delivery system of any of claims 1-6 further comprising one or more emulsifier(s), oil(s) and/or active ingredient(s).

8. A topical probiotic delivery system of any of claims 1-7 that is substantially anhydrous.

9. A topical probiotic delivery system of claim 8 that is substantially free of preservatives.

10. A cream, lotion, serum, mask or spray for application to a keratinous substrate comprised of (i) a topical probiotic delivery system of any of claims 1-9 and (ii) a non-cytotoxic, non-bacteriostatic liquid carrier.
